(19) United States
(12) Patent Application Publication
(10) Pub. No.: US 2023/0363781 A1
(43) Pub. Date: Nov. 16, 2023

(54) SURGICAL TOOL, SYSTEM AND METHOD

(71) Applicant: ACADEMISCH ZIEKENHUIS LEIDEN (H.O.D.N. LUMC), Leiden (NL)

(72) Inventors: Dhr. Matthias VAN OOSTEROM, Leiden (NL); Dhr. Simon VAN ALPHEN, Leiden (NL); Dhr. Fijs VAN LEEUWEN, Leiden (NL); Dhr. Michael BOONEKAMP, Leiden (NL); Dhr. Jeroen VERHART, Leiden (NL)

(21) Appl. No.: 18/030,356

(22) PCT Filed: Oct. 11, 2021
(86) PCT No.: PCT/NL2021/050616

§ 371 Date: Apr. 5, 2023

(30) Foreign Application Priority Data
Oct. 9, 2020 (NL) 2026652
Apr. 9, 2021 (NL) 2027947

(51) Int. Cl. A61B 17/29

(52) U.S. Cl. CPC A61B 17/29; A61B 2017/00477

(57) ABSTRACT

A mounting device for releasably coupling a detector to a surgical instrument of the type comprising first and second jaws displaceably mounted with respect to each other, each jaw comprising a grasping surface for grasping subject tissue in a grasping region defined between the jaws, comprises: detector mounting means configured for releasable mounting and retention of a detector; and coupling means configured for releasably coupling the detector mounting means to a jaw of the surgical instrument to position the detector mounting means outside the grasping region.

The present invention relates to a surgical tool, system and method for use in surgery.

### BACKGROUND

In the surgical management of cancer, for example prostate cancer, robot-assisted laparoscopic surgery is now a routine procedure. Robot-assisted surgery offers many advantages, but it can also create challenges, for example when attempting to perform radioguided surgery using a rigid laparoscopic gamma probe to identify radioactive emissions from target tissue which has absorbed a radioactive tracer injected into the patient’s body. The limited manoeuvrability of such a probe restricts identification of lesions, especially when low-activity lesions are situated in close proximity to a high-activity background. In robot-assisted (master-slave) surgery, where the surgical console is placed away from the patient bed, the surgeon relies on the bedside assistant for the positioning of the laparoscopic gamma probe, which is something that he/she has to verbally guide.

With the aim of regaining autonomy and increasing manoeuvrability during radioguidance, it is known to allow the robotic tools to position a tethered probe e.g. a gamma probe, for detecting radioactive emissions from target tissue which has absorbed a radioactive tracer. In this arrangement, the bedside assistant inserts the gamma probe through a trocar into the abdominal cavity where it can be picked up by the surgeon using the robotic surgical instruments. The surgeon can leave the gamma probe within the abdominal cavity during the procedure and can pick up the probe whenever he or she needs to search for radioactive lesions. It is also known to use other types of probe, for example a probe for detecting beta particles or an ultrasonic transceiver.

However, picking up the probe in a slippery soft-tissue environment such as an abdominal cavity can be difficult, and the necessity for picking up the gamma probe in order to identify radioactive lesions and then putting down the probe after identification, usually several times during a surgical procedure, can significantly increase the overall length of the procedure.

### BRIEF SUMMARY OF THE INVENTION

In accordance with a first aspect of the present invention, a mounting device for releasably coupling a detector to a surgical instrument of the type comprising first and second jaws displaceably mounted with respect to each other, each jaw comprising a grasping surface for grasping subject tissue in a grasping region defined between the jaws, comprises:
detector mounting means configured for releasable mounting and retention of a detector; and
coupling means configured for releasably coupling the mounting device to a jaw of the surgical instrument to position the mounting device outside the grasping region.

By having a separate detector mounting means on which the detector can be releasably mounted and retained, and which itself can be releasably coupled to a surgical instrument, it is possible to releasably secure the detector to the surgical instrument, which allows identification of radioactive lesions without having to pick up and put down the detector and which also allows the surgical instrument to perform its normal tasks. This can significantly reduce the overall time taken for a surgical procedure.

In addition, by having a separate detector mounting means, it is possible to configure the detector mounting means to be disposable, if desired, which reduces the likelihood of cross contamination and infection. It also removes the need to configure the shape of the detector to match that of the surgical instrument, which might otherwise compromise the operation of the detector.

The detector mounting means and the detector may be configured to be a releasable interference fit with each other.

This allows, in a first mode of use, the detector to be releasably fitted to the detector mounting means and the detector mounting means in turn to be releasably fitted to a surgical instrument, such as a pair of forceps. This can be done externally of the body of the patient undergoing surgery and then the assembly can be inserted into the body of the patient via a trocar. The position of the detector can then be adjusted by altering the position of the surgical instrument to which the detector is attached. This way the flexibility of the surgical instrument helps to optimally support the identification of tissue material, which produces emissions (e.g. radioactive emissions) which the detector is configured to detect. As the function of the surgical tool (e.g forceps) is maintained, it can then immediately be used in the surgical process without having to manoeuvre it to first set down the detector and reposition the tool for surgery.

The detector mounting means may comprise a recess for receipt of the detector.

The surgical tool may comprise an elongate recess for receipt of the detector.

The recess may be defined between two spaced apart side walls.

The side walls may be elongate.

The side walls are preferably substantially parallel.

The side walls may be resiliently deformable to permit the detector to be inserted into the recess and retained in the recess by the side walls.

The detector mounting means may be configured to receive the detector as a press fit.

The coupling means may be configured for releasably coupling the mounting device to an outer surface of a jaw.

A portion of the detector mounting means is preferably shaped to be an interference fit with a portion of a jaw of the surgical instrument.

For example, a portion of the coupling means may be shaped to fit with a complementarily-shaped portion of a jaw of the surgical instrument.

The coupling means may comprise a projection which is shaped to fit with a complementarily shaped portion of a jaw of the surgical instrument.

The projection may be elongate.

In one embodiment, the projection is located between two side walls of the detector mounting means.

A portion of the coupling means may be shaped to fit with a complementarily-shaped portion of a jaw of the surgical instrument, for example a jaw of a pair of forceps.

The coupling means may further comprise one or more portions shaped to facilitate gripping by a surgical instrument.

This allows the surgical tool to be used in a second mode of operation in which the detector is mounted on the detector mounting means and inserted into the interior of a patient’s body via a trocar. A surgical instrument (e.g. a pair of forceps) can then be used to pick up the detector by means of the detector mounting means as and when required, in order to adjust the position of the detector so as to identify emissive target tissue which the detector is configured to detect. The provision of one or more portions of the detector mounting means which are shaped to facilitate gripping by a surgical instrument greatly facilitates the second mode of use, in the slippery soft-tissue environment within the body

The coupling means may comprise one or more portions complementarily shaped with a jaw of a pair of forceps.

The coupling means may comprises one or more recesses for receipt of a portion of a jaw of a pair of forceps.

The or each shaped portion may be located in a side wall of the detector mounting means.

In accordance with a second aspect of the present invention, a surgical tool comprises a mounting device in accordance with the pesent invention, in combination with a detector configured for releasable mounting and retention by the detector mounting means.

The detector may be elongate.

The detector may have a detection axis which is aligned with a generally longitudinal direction of the surgical instrument.

In accordance with a third aspect of the present invention, a surgical tool for detecting an emission from a subject tissue comprises first and second jaws displaceably mounted with respect to each other, each jaw comprising a grasping surface for grasping subject tissue in a grasping region defined between the jaws, a housing mounted on one of the jaws and a detector enclosed within the housing for detecting the emission from the subject tissue, wherein the housing comprises a portion facing the grasping surface of the opposite jaw which permits the passage of emissions being detected by the detector.

In a preferred embodiment, the housing substantially prevents the passage of emissions being detected by the detector except for the portion of the housing facing the grasping surface of the opposite jaw which permits the passage of emissions being detected by the detector.

In one embodiment, the portion of the housing facing the grasping surface of the opposite jaw which permits the passage of emissions being detected by the detector is elongate.

The portion of the housing facing the grasping surface of the opposite jaw which permits the passage of emissions being detected by the detector may comprise a collimation slit.

Preferably, the opposite jaw substantially prevents the passage of emissions being detected by the detector.

This ensures that background radioactive signals from below the exterior of the jaw opposite the detector are not detected by the detector.

The jaws may be pivotally mounted with respect to each other.

The surgical tool may further comprise a transmission device coupled to the detector, the transmission device being configured to transmit a signal to a signal processing device, the signal being a function of the emission as detected by the detector.

The surgical tool may further comprise a cable connecting the transmission device to the signal processor.

The detector may be configured to detect radioactive emissions.

For example, the detector may comprise a gamma ray detector.

In accordance with a fourth aspect of the present invention, a surgical system comprises a surgical tool in accordance with the second aspect of the present invention, and a surgical instrument.

The surgical instrument may comprise a pair of forceps.

The surgical system may comprise means for outputting an audible and/or visual indication of the emissions detected by the detector. Depending on the detector, the surgical system may also comprise means for displaying an image or images, for example if the detector is an untrasonic transceiver.

The surgical system may be configured for use during laparoscopic surgery.

For example, the surgical system may be configured for use during robot-assisted laparoscopic surgery.

In accordance with a fifth aspect of the present invention, a method of performing tissue visualisation and characterisation comprises use of the mounting device in accordance with the first aspect of the invention and/or the surgical tool in accordance with the second aspect of the invention and/or the surgical system in accordance with the third aspect of the invention.

### BRIEF DESCRIPTION OF THE DRAWINGS

By way of example only, specific embodiments of the present invention will now be described with reference to the accompanying drawings, in which:

FIGS. 1(a) and 1(b) are perspective views, from different angles, of a first embodiment of surgical tool in accordance with the present invention, shown coupled to surgical forceps;

FIG. 2 is a perspective view of the surgical tool and surgical forceps of FIG. 1, showing the surgical forceps extending from a robotic surgical instrument;

FIGS. 3(a) and 3(b) are exploded perspective views of the surgical tool and surgical forceps shown in FIG. 1;

FIG. 4 shows the surgical tool and surgical forceps of FIG. 1 locating a radioactive subject tissue;

FIGS. 5(a) and (b) are perspective views, from different angles, of the surgical tool and surgical forceps of FIG. 1, shown in a different configuration;

FIG. 6 is a perspective view of a second embodiment of surgical tool in accordance with the present invention;

FIG. 7 is an expoloded perspective view of the surgical tool of FIG. 6; and

FIG. 8 is a view from below of the housing of the surgical tool of FIG. 6.

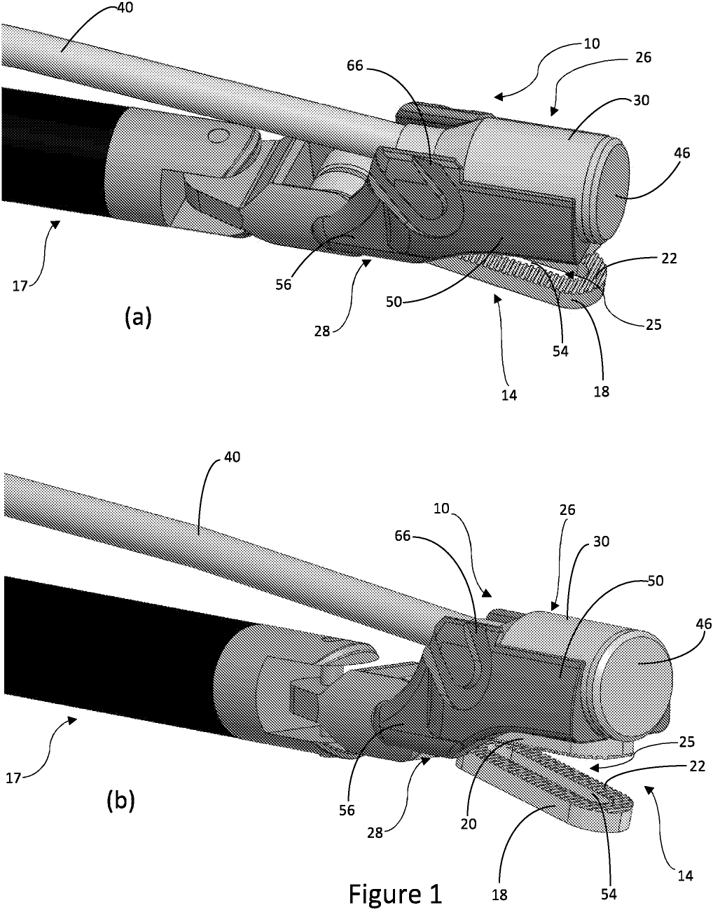

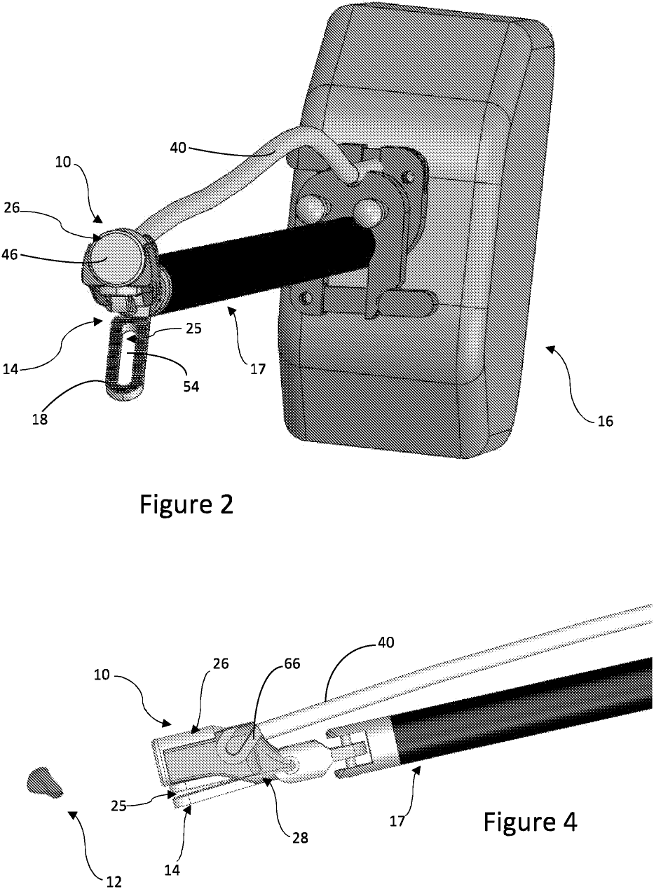

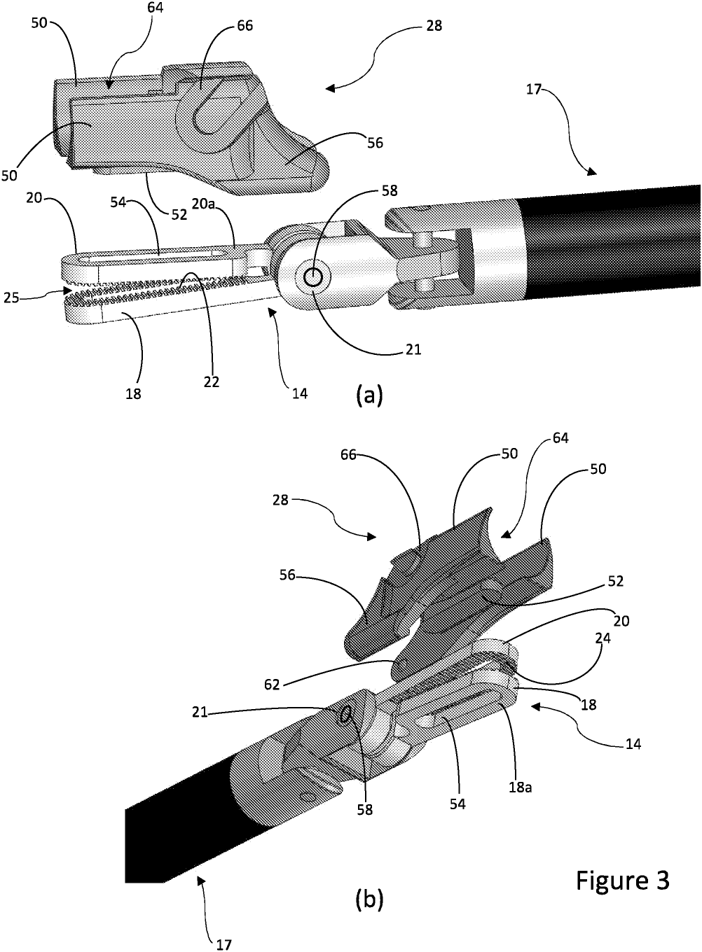

FIGS. 1 to 5 illustrate a first embodiment of surgical tool 10 in accordance with the present invention, which relates to sensing of gamma rays emitted from subject tissue 12 (see FIG. 4) which has been rendered artificially radioactive by injection of radioactive material into the patient which is subsequently taken up by the subject tissue 12. FIGS. 1 and 3 show a surgical instrument 16, e.g. an instrument of a da Vinci ® surgical robot system manufactured by Intuitive, Inc., having a pair of surgical forceps 14 located at a distal end of an adjustable arm 17 which forms part of the surgical instrument 16. The surgical tool 10 is releasably secured to the forceps 14, as will be explained., e.g. ProGrasp ® forceps.

The surgical forceps 14 comprise a pair of jaws 18, 20 pivoted about a pivot pin 21, with each jaw 18, 20 having a respective working or grasping surface 22, 24 intended for grasping biological tissue such as the subject tissue 12 shown in FIG. 4 in a grasping region 25 between the working (grasping) surfaces 22, 24 of the jaws 18, 20. The forceps 14 themselves have no means by which tissue can be identified. However, the surgical tool 10 comprises means for determining tissue characteristics, as will be explained. Therefore, coupling of the surgical tool 10 to the forceps 14 provides the forceps (or other suitable surgical instrument) with the capability to readily identify radioactive hotspots within a patient’s body.

The surgical tool 10 comprises a detector 26 for detecting an emission from subject tissue 12 grasped between the working surfaces 22, 24 of the jaws 18, 20, within the grasping region 25, and a mounting means or holder 28 on which the detector 26 is releasably mounted. The detector mounting means or holder 28 is in turn releasably coupled to the outer surface or face 20a of one of the jaws 20 of the forceps 14, so that the detector mounting means or holder 28 and the detector 26 lie outside the grasping region 25, as will be explained. It should also be noted that the detector mounting means or holder 28 may instead be releasably coupled to the outer surface or face 18a of the other of the jaws 18 of the forceps 14. In this particular embodiment, the detector 26 is a gamma ray detector, but other detectors (e.g. beta particle detectors) may be used instead, depending on the emission properties of the subject tissue.

The detector 26 illustrated is largely conventional and comprises a tubular tungsten outer housing 30 (although other high-density metals such as gold, platinum or platinum-iridium alloy could be used) which in this embodiment encloses a conventional gamma ray detector (not visible), although the nature of the detector will depend on the type of radiation to be detected. A cable 40 passes sealingly through the rear end of the outer housing 30 and the front end of the outer metal housing 30 is sealed by means of a disc-shaped titanium front seal 46. Electrical signals from the detector are transmitted along the cable 40 to an external controller (not shown) for determining the detected gamma ray activity and for subsequent transmission to a user interface on a surgical or read out console configured to display the tissue characteristics in dependence on the detected emission, in a given output form such as a numerical, graphical or audible output or a combination of two or more outputs.

The mounting means or holder 28 is formed from a plastics material such as polyether ether ketone (PEEK), polyoxymethylene (POM), nylon or other suitable plastics material, and is intended to be disposable, if desired. The holder 28 is preferably moulded and comprises two elongate, parallel, spaced apart, lateral side walls 50 which are mirror images of each other. The side walls 50 joined by an elongate base member 52 extending parallel to the longitudinal direction of the side walls 50 and which is shaped into an elongate oval projection which in use is received in a complementarily-shaped recess 54 provided in each of the jaws 18, 20 of the surgical forceps 14. The rear end of each of the side walls 50 is formed into a hemispherical lug 56 which projects beyond the level of the base member 52. When the elongate projection 52 of the holder 28 is received in the recess 54 of one of the jaws 18, 20 of the surgical forceps, the lugs 56 extend over the pin 21 about which the jaws 18, 20 are pivoted and the inner face of each of the projecting lugs 56 is provided with a projection 62 which engages with, and is received in, a complementarily-shaped cavity 58 in the end face of the pivot pin 21, thereby further releasably retaining the mounting means or holder 28 on the forceps 14.

The side walls 50 of the holder 28 are arcuate in transverse cross-section and define between them an elongate recess 64 which is complementarily-shaped with the outer surface of the detector 26. Because of the flexible and resiliently deformable nature of the detector holder 28, the detector 26 can be press fitted into the recess 64 by deforming the walls 50 outwardly, after which the detector 26 is securely held in place by the walls 50 of the holder. Removal of the detector from the holder is achieved by simply pulling the detector out of the recess 64, which temporarily deforms the side walls 50 outwardly. The longitudinal axis of the recess and the detector 26 are aligned with the general longitudinal direction of the forceps (or other surgical instrument) and is thus conveniently positioned to “probe” for radioactivity whilst keeping the forceps available for use as normal.

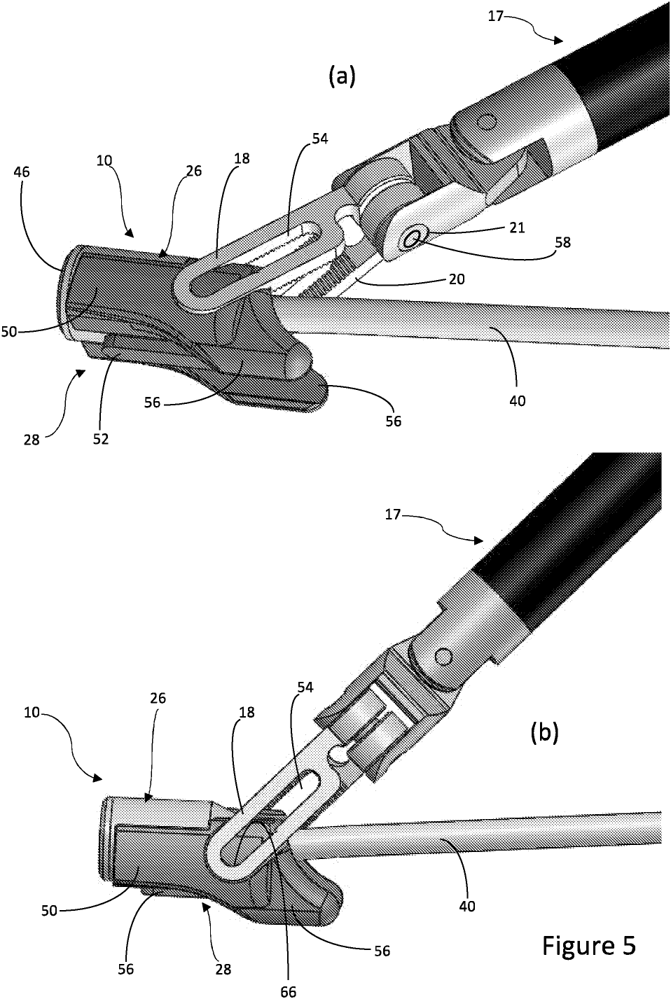

It will also be observed that the outer face of each of the side walls 50 is provided with a U-shaped recess 66 which is complementarily-shaped with, and is intended to receive, the end of a respective one of the jaws 18, 20 of the pair of forceps 14 for use in a different configuration, which will be explained later with reference to FIG. 5.

In a first mode of use, the detector 26 is securely, but releasably, mounted on the holder 28 by press fitting it into the recess 64 as described above. The holder 28 is then coupled, also securely but releasably, to the outer surface or face 20a of one of the jaws 20 of the surgical forceps 14 by locating the elongate recess 52 in the base of the holder 28 in the complementarily-shaped recess 54 in one of the jaws 20 of the pair of forceps 14. Alternatively, the holder 28 could be coupled to the outer surface or face 18a of the other of the jaws 18 of the forceps 14. The assembled surgical instrument consisting of the forceps 14, detector 26 and holder 28 is then inserted into the body of a patient (into the abdominal cavity for prostate surgery) via a trocar.

As illustrated in FIG. 4, the surgical forceps 14 and the detector 26 which is mounted on the forceps via the holder 28 can be manoeuvred to search for tissue which has been stained with a radioactive tracer injected into the patient’s body. If the subject tissue 12 has absorbed the radioisotope tracer, it will produce radioactive emissions (in this case gamma rays) which can be detected by the detector 26 (as indicated by the user interface to which the output cable 40 is connected) in order to identify the tissue which is to undergo surgery. As the detector 26 moves with the surgical forceps 14, and as the detection axis of the detector 26 is aligned with the longitudinal direction of the forceps 14, it is possible to “probe” for radioactive emissions and it is not necessary to reposition the detector 26 after the correct subject tissue has been identified before the forceps can be used in the surgical procedure. The detector 26 and its holder 28 are mounted outside the grasping region 25 of the forceps 14 and are not grasped by the jaws 18, 20 of the forceps 14. Consequently, normal operation of the forceps is not inhibited by the mounting of the detector 26 and its holder 28 on one of the jaws, and the forceps 14 can continue to be used as forceps in the normal way with the detector 26 and its holder 28 in place, without the need to first set down the detector to allow the forceps 14 to be used. This can significantly reduce the overall time of surgical procedures and can also minimise the likelihood of the incorrect subject tissue being operated on.

In a second mode of use, the detector 26 is securely, but releasably, mounted on the holder 28 by pressing it into the recess 64 as described above. The surgical tool consisting of the detector 26 and holder 28 is then inserted into the body of a patient (into the abdominal cavity for prostate surgery) via a trocar. The surgical forceps 14 can then be inserted into the patient via the trocar and, when desired, can be used to pick up the detector 26 and holder 28 to “search” for a radioactive lesion. In this mode of operation, the jaws 18, 20 of the surgical forceps are engaged with, and received in, the complementarily-shaped recesses 66 in the outer face of each of the two side walls 50 of the holder 28, which facilitates picking up of the detector 26 and holder 28 in a slippery soft-tissue environment within the body. In this mode of operation, detector 26 is used to probe for radioactive emissions from potential subject tissue 12 only as and when required, which can be desirable in some circumstances.

The provision of a separate detector mounting device, to which the detector can be attached and which can be attached to a surgical instrument, allows the detector mounting means to be disposable, if desired. However, although in the specific embodiment described the detector mounting device is made of plastics material, other materials such as metal can be used, particularly if it is intended that the detector mounting device should be capable of being reused.

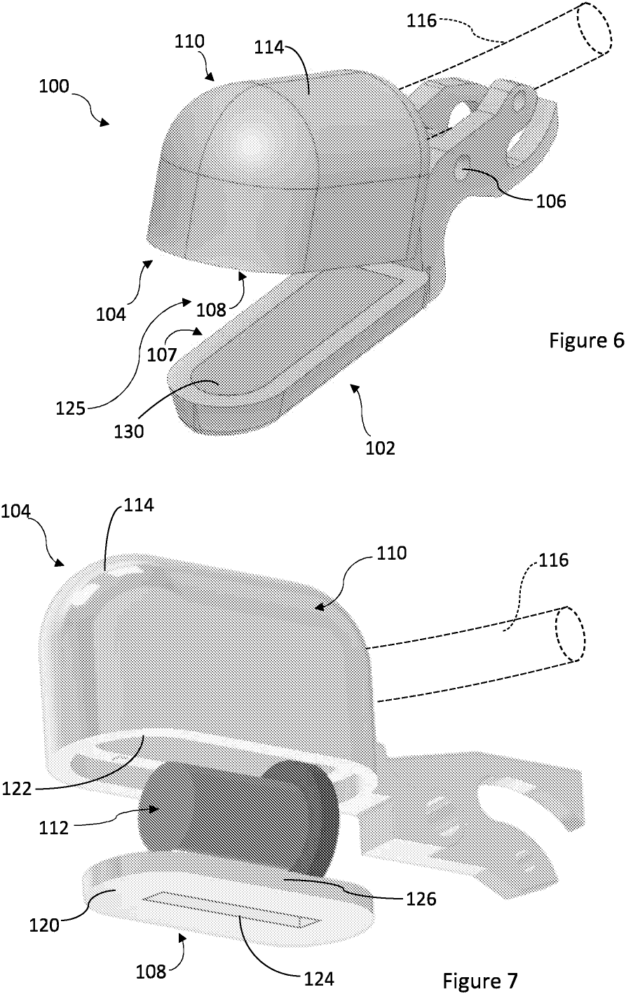

A second embodiment of the present invention is illustrated in FIGS. 6 to 8. In the first embodiment, the detection axis is aligned with a generally longitudinal direction of the surgical instrument, so that the detector can be used to “probe” for radioactive emissions. In contrast, the second embodiment is designed to detect radioactive emissions in the tissue being gripped.

The second embodiment comprises surgical forceps 100 having a first, lower jaw 102 and a second, upper jaw 104 pivoted about a pivot pin 106, and which is configured to be attached to a surgical robot system (not illustrated) as in the first embodiment. The lower and upper jaws 102, 104 have gripping surfaces indicated generally at 107, 108 which face each other and between which a grasping region 125 is defined.As will be explained, the outer surface or face of the upper jaw 104 carries a detector 112 for detecting radioactive emissions from tissue held in the grasping region 125 between the gripping surfaces 107, 108 of the lower and upper jaws 102, 104.

The upper jaw comprises an elongate housing 110 within which a detector 112 for radioactive emissions is enclosed. In the particular embodiment illustrated, the detector 112 is identical to the gamma ray detector 26 of the first embodiment but a different detector or detectors, or one or more detectors sensitive to radiation other than gamma ray radiation may be used.

The housing 110 is formed from a moulded plastics material but has one or more inner layers (not visible) which render the housing opaque to the radiation to be detected (i.e. substantially prevents the passage of the radiation to be detected), and comprises a bulbous upper surface 114 to contain the detector 112. As in the first embodiment, a cable shown schematically at 116 passes sealingly through an aperture 118 at the end of the housing 110 adjacent to the pivot pin 106. Electrical signals from the detector are transmitted along the cable 116 to an external controller (not shown) for determining the detected gamma ray activity and for subsequent transmission to a user interface on a surgical or read out console configured to display the tissue characteristics in dependence on the detected emission, in a given output form such as a numerical, graphical or audible output or a combination of two or more outputs.

The lower face of the housing 110, which opposes the lower jaw 102, is flat and is formed from a removable straight-sided elongate oval plate 120 which is releasably securable in a complementarily-shaped recess 122 in the lower face of the housing. The depth of the recess 122 is the same as the depth of the removable plate 120 so that the plate 120 lies flush with the undersurface of the housing 110 when secured in position, thereby forming the gripping surface 108 of the upper jaw 104.

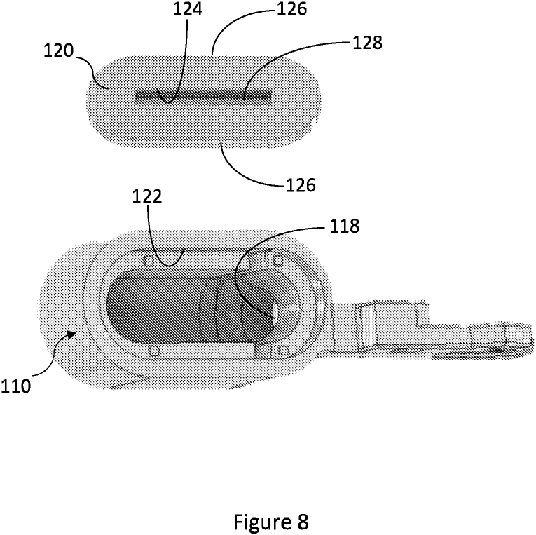

The elongate oval plate 120 is formed from material which is opaque to the radiation being detected (i.e. substantially prevents the passage of the radiation to be detected), except for a narrow elongate straight collimation slit 124 which extends parallel to the longitudinal axis of the plate 120 and is located equidistant from the two straight sides 126 of the plate, as shown in FIGS. 7 and 8. The collimation slit 124 has a base wall 128 which isolates the interior of the housing 110 from the undersurface of the housing 110, but which is (mostly) transparent to the radioactivity to be detected.

The lower jaw 102 is provided with a clamping surface 130 which is configured to engage body tissue under investigation. It houses an elongated oval collimation plate which blocks radioactive signal originating from below the lower jaw to ensure that radioactivity measured with the detector only originates from within the jaw when the tissue is grasped, and not from outside the jaw gripping surfaces.

In use, the forceps 100 are inserted into the body of a patient (for example into the abdominal cavity for prostate surgery) via a trocar. As mentioned previously, the forceps 100 are attached to a surgical robot system (not illustrated) as in the first embodiment and are manoeuvred within the patient’s body to search for tissue which has been stained with a radioactive tracer injected into the patient’s body.

In contrast to the first embodiment, the surgeon operating the forceps 100 can grip a portion of tissue under investigation located in the grasping region 125 between the lower and upper jaws 102, 104 of the forceps and a direct measurement of the radioactivity of the gripped portion of tissue can be made by the detector 112 via the collimating slit 124. In this way, the surgeon operating the equipment can be certain that the measured radioactivity corresponds to that of the gripped portion of tissue, in contrast to the first embodiment in which detection of radioactive tissue is made to the front of the instrument, facilitating ‘probing’ of the tissue of interest during the search process (instead of ‘grabbing’ the tissue of interesting during confirmation of the radioactive tissue).

The invention is not restricted to the details of the foregoing embodiments.

For example, the embodiments make reference to a detector for gamma rays. However, depending on the subject tissue 12, the radioactive tracer selected and other factors, detectors for other types of radioactivity, for example beta particles, might be used instead. The invention is also applicable to detection of radioactive location “seeds”.

In the specific embodiments, the electronic components of the detector are located within the detector housing. As an alternative, a different type of detector may be used, in which the electronic components are located remotely, outside the patient’s body.

However, the invention is not restricted to detection of radioactivity. For example, the detector may comprise an ultrasonic or optical transceiver which is configured to produce a visual image of the tissue under investigation, or may comprise a detector configured to detect a fluorescent tracer illuminated by a suitable radiation source which induces fluorescence (e.g. an ultraviolet radiation source).

1. A mounting device (28) for releasably coupling a detector (26) to a surgical instrument (14) of the type comprising first and second jaws (18, 20) displaceably mounted with respect to each other, each jaw (18, 20) comprising a grasping surface (22, 24) for grasping subject tissue (12) in a grasping region (25) defined between the jaws, the mounting device comprising:
detector mounting means (50, 64) configured for releasable mounting and
retention of a detector (56); and
coupling means (52, 54) configured for releasably coupling the mounting device (28) to a jaw (18, 20) of the surgical instrument (14) to position the mounting device outside the grasping region (25).

2. A mounting device as claimed in claim 1, wherein the detector mounting means (50, 64) is configured to be a releasable interference fit with a detector (56).

3. A mounting device as claimed in claim 2, wherein the detector mounting means comprises a recess (64) for receipt of the detector (56).

4. A mounting device as claimed in claim 3, comprising an elongate recess (64) for receipt of the detector.

5. A mounting device as claimed in claim 3, wherein the recess (64) is defined by two spaced apart side walls (50).

6. A mounting device as claimed in claim 5, wherein the side walls (50) are elongate.

7. A mounting device as claimed in claim 6, wherein the side walls (50) are substantially parallel.

8. A mounting device as claimed in claim 5, wherein the side walls (50) are resiliently deformable to permit the detector (56) to be inserted into the recess (64) and retained in the recess by the side walls (50).

9. A mounting device as claimed in claim 2, wherein the detector mounting means (50, 64) is configured to receive the detector (56) as a press fit.

10. A mounting device as claimed in claim 1, wherein the coupling means (52, 64) is configured for releasably coupling the mounting device to an outer surface of a jaw (18, 20).

11. A mounting device as claimed in claim 1, wherein a portion (52) of the coupling means (50, 64) is shaped to be an interference fit with a portion (54) of a jaw (18, 20) of the surgical instrument (14).

12. A mounting device as claimed in claim 1 preceding, wherein a portion (52) of the coupling means (50, 64) is shaped to fit with a complementarily-shaped portion (54) of a jaw (18, 20) of the surgical instrument (14).

13. A mounting device as claimed in claim 12, wherein the coupling means comprises a projection (52) which is shaped to fit with a complementarily shaped portion (54) of a jaw (18, 20) of the surgical instrument (14).

14. A mounting device as claimed in claim 13, wherein the projection (52) is elongate.

15. A mounting device as claimed in claim 13, wherein the projection (52) is located between two side walls (52) of the detector mounting means.

16. A mounting device as claimed in claim 11, wherein a portion (52) of the coupling means is shaped to fit with a complementarily-shaped portion (54) of a jaw (18, 20) of a pair of forceps (14).

17. A mounting device as claimed in claim 1, wherein the coupling means further comprises one or more portions (66) shaped to facilitate gripping by a surgical instrument.

18. A mounting device as claimed in claim 17, wherein the coupling means comprises one or more portions (66) complementarily shaped with a jaw of a pair of forceps.

19. A mounting device as claimed in claim 18, wherein the coupling means comprises one or more recesses (66) for receipt of a portion of a jaw of a pair of forceps.

20. A mounting device as claimed in claim 17, wherein the or each shaped portion (66) is located in a side wall (52) of the detector mounting means.

21-39. (canceled)
